(19) United States
(12) Patent Application Publication
(10) Pub. No.: US 2025/0060339 A1
(43) Pub. Date: Feb. 20, 2025

(54) ACOUSTIC VIBRATIONS METHOD TO DETECT DISEASES AND PREDICT SHELF LIFE AND MATURITY OF COMMODITIES

(71) Applicant: Shubham Chandra, Katy, TX (US)

(72) Inventor: Shubham Chandra, Katy, TX (US)

(21) Appl. No.: 18/318,719

(22) Filed: May 17, 2023

(51) Int. Cl. G01N 29/24; B25J 19/02; G01N 29/04; G01N 29/265; G01N 29/44; G01N 33/02

(52) U.S. Cl. CPC G01N 29/2437; B25J 19/028; G01N 29/04; G01N 29/265; G01N 29/44; G01N 33/025; G01N 2291/023; G01N 2291/0289

(57) ABSTRACT

A non-destructive method to detect the onset of diseases in organic cells and predict shelf life and maturity of commodities including fruits, vegetables, seeds, meat, fish, freezer dried products, beverages and pharmaceutical drugs is presented here. The system includes a bone conduction speaker, piezoelectric sensor-based microphone, and an audio processor (including shelf-life matrix, diseases matrix, defect matrix and maturity matrix specific to each perishable commodity) and a display system, which automatically determines ready for harvest condition, diseases (if any), maturity and the remaining shelf life of the perishable commodity.

### BACKGROUND

### Technical Field

The present disclosure relates to an acoustic method that can be used to detect the onset of diseases in organic cells and predict shelf life and maturity of commodities including fruits, vegetables, seeds, meat, fish, freezer dried products, beverages and pharmaceutical drugs. Acoustic waves can be used to measure the properties of fruits and vegetables and other perishables, such as stiffness, density, and moisture content. These properties are related to the shelf life and maturity of these perishable commodities. It is also possible to detect diseases in plants and humans using acoustic emission, that works by detecting sound waves that are produced when plants/cells are damaged. In lieu of this patent application and research, no federally/Government sponsored research or development-based funding was used.

### Technical Background

According to the Food and Agriculture Organization of the United Nations (FAO), about 1.3 billion tons of food is wasted globally every year. This represents about ⅓ of all food produced for human consumption. The majority of food waste occurs at the production and distribution stages, while a smaller amount is wasted at the consumption level. Food waste has a number of negative environmental and social impacts. It contributes to climate change, as methane, a greenhouse gas, is released when food decomposes in landfills or incinerators. Food waste also consumes resources, such as water and energy, and it contributes to deforestation, as land is cleared to grow more food. There are a number of things that can be done to reduce food waste. At the production and distribution stages, food waste can be reduced by improving harvesting and storage techniques, as well as by reducing transportation losses. At the consumption level, food waste can be reduced by educating consumers on how to properly store and cook food, and by encouraging them to donate food that would otherwise be wasted.

While most of these food losses are happening at the farmgate, distribution and retail stores, the food waste is primarily happening at the consumption stage. The shelf life, defined as the length of time that a commodity may be stored without becoming unfit for use, consumption, or sale, becomes of paramount importance. Maturity is considered as one of the most important quality determination factors in harvesting at the right time and for the postharvest handling of produce. The capability to know the shelf life will lead to better decision making. Batches which have a longer shelf life, could be used for exports and longer transit markets, while those with shorter shelf life could be directed to more localized markets. This understanding of the shelf life of the produce will help in preventing these losses and wastages, reducing costs, and improving overall freshness. Perishable commodities, such as fruits, vegetables, seeds, freeze dried products, meats, fish, and pharmaceutical products each have a unique shelf life.

The shelf-life prediction methods collect data and subsequently use algorithms to estimate the time to reach end of shelf life. Some of this data is based upon the following parameters:
1. Brix value
2. Ethylene generated
3. Taste
4. Bruising intensity
5. Firmness
6. Color
7. Visual defects
8. Initial growing conditions
9. Temperature
10. Humidity
11. Smell
12. Chemical secretions including Indoleacetic Acid (IAA), Jasmonate, and Ethylene

The hormone that is released when a fruit begins to mature is indoleacetic acid (IAA). IAA is a plant hormone that is involved in many aspects of plant growth and development, including cell division, elongation, and flowering. It is also involved in the ripening of fruits.

IAA is released from the growing tip of the fruit and travels throughout the fruit, where it stimulates the production of ethylene. Ethylene is the gas that is responsible for many of the changes that occur during ripening, such as the softening of the fruit, the change in color, and the development of flavor.
IAA is also involved in the development of the fruit's seed. It stimulates the growth of the seed and the production of the embryo.

Another chemical that is released when a fruit begins to mature is Jasmonate. Jasmonate is a plant hormone that is produced in response to a variety of stimuli, including stress, infection, and wounding. It has a variety of effects on plants, including the induction of defense responses, the suppression of growth, and the stimulation of flowering.

The methods for shelf-life prediction can be categorized into two main categories:
a. Destructive Methods (Chemical Analysis (including sucrometer readings/Static Testing)
b. Non-Destructive Methods (Temperature/Humidity Monitoring, Imaging including hyperspectral, Spectroscopy, Magnetic Resonance Imaging, and acoustic vibration technology). Other techniques include, electronic sniffing, ultrasonic testing, optoclectrics, and thumping etc.

Here is a detailed description of some of the Non-Destructive Methods:
Spectroscopy: This method uses light to analyze the chemical composition of a fruit or vegetable. The results can be used to predict the shelf life of the food. Ultraviolet spectroscopy can be used to detect shelf life of fruits by measuring the changes in the absorption of ultraviolet light by the fruits as they ripen. The absorption of ultraviolet light by fruits is due to the presence of compounds called pigments, which give fruits their color. As the fruits ripen, the pigments break down and the absorption of ultraviolet light decreases. This decrease in absorption can be used to determine the shelf life of the fruits.
Near-infrared spectroscopy: This method is similar to spectroscopy, but it uses near-infrared light instead of visible light. Near-infrared light is absorbed by water molecules, so it can be used to measure the moisture content of a fruit or vegetable. The moisture content is a good indicator of the shelf life of the food.
Electrical conductivity: This method measures the electrical conductivity of a fruit or vegetable. The results can be used to predict the shelf life of the food.
Ultrasound: This method uses ultrasound to create an image of the inside of a fruit or vegetable. The image can be used to identify areas of damage or decay.
Machine learning: This method uses artificial intelligence to analyze data from sensors or other sources. The results can be used to predict the shelf life of a fruit or vegetable.
Nuclear magnetic resonance (NMR): NMR is a technique that uses a magnetic field to measure the properties of a substance. It can be used to measure the water content, fat content, and protein content of fruits and vegetables.

There are several challenges associated with shelf-life prediction methods, some of these challenges include:
The variability of fruits. Fruits are living organisms that are constantly changing. This makes it difficult to develop a model that can accurately predict the shelf life of all fruits.
The complexity of fruit tissues. Fruits are complex tissues that are made up of a variety of different components. This makes it difficult to identify the specific factors that are responsible for the changes that occur in fruits as they ripen.
The lack of accurate data. There is a lack of accurate data on the shelf life of fruits. This makes it difficult to develop a model that can accurately predict the shelf life of fruits.
   Inconsistent cold chain, lack of postharvest procedures, inconsistent ethylene ripening techniques, and inconsistent quality procedures, are all the challenges associated with predicting the shelf life of the fruit/vegetables.

At this time there is no accurate non-destructive way to predict the actual remaining shelf of a produce commodity and the maturity of the produce. Fruit quality related to ripeness has many indicators, such as appearance, sugar content, acid content, internal defeet, and firmness. Among these indices, fruit firmness is closely related to the fruit's physical structure, and properties, which are very important at different stages of the food supply chain. At the harvest stage, firmness is used for predicting the optimal harvest time. During sorting and grading, firmness serves as the basis of the sorting strategy. In distribution, firmness helps in defining the optimal transport conditions and packaging solutions. Firmness is also utilized to estimate the optimum edibility and shelf life. Currently, fruit firmness is measured using destructive methods such as Magness-Taylor (MT) puncture test and firmness test. The puncture test measures the force required for a probe to penetrate into a sample for a specific depth. The compression test records the force signal under crushing loads using a flat plate. The major disadvantages of these methods are, they are: time consuming, destructive, non-repeatable, labor intensive, local and equipment dependent.

With these variabilities in the supply chain into perspective, it is very difficult to commercially specify the shelf life and freshness of fruits and vegetables. This method optimizes the management of the fruits and vegetables/meat supply chain, by empowering the farmers, distribution centers, retails stores, and the home consumers by providing with critical remaining shelf-life information, to make informed decisions, which ensures reduction in supply chain risks, reduction in food waste and losses, and overall reduction in carbon footprint. Components of this methodology can be cither embedded in robotics systems/autonomous drones at the farmgate, allowing for easier prediction of harvest dates, and reduction in losses on the tree/plant of fruits and vegetables, and eventual retrieval of the same. Such methods will be useful in accurately predicting the shelf life of food items, when embarking on long duration missions such as space travel. Certain components of this methodology can also be used for finding anomaly detection. All things in our universe, including perishables such as fruits and vegetables, are constantly in motion, vibrating. Even objects that appear to be stationary are in fact vibrating, oscillating, resonating, at various frequencies. All objects have natural frequencies and vibrate more easily at these specific frequencies. When an object vibrates at its natural frequency, this is called resonance. Sounds is a vibration that typically propagates as an audible wave of pressure in the range of 20 to 20,000 hertz through a transmission medium such as a gas, liquid, or solid. The physical properties of the medium have a great influence on the acoustic characteristics, such as the sound velocity, frequency, and sound pressure. When sound travels through a fruit, it will interact with the internal tissues of the fruit. The propagation velocity shall be affected by the density and Young's modulus of the fruit, which is directly related to the firmness of the fruit. Generally, the relationships of the propagation velocity with the density and clastic modulus of a fruit can be expressed as below:

$$v = \sqrt[2]{\frac{E}{2 p ( 1 + u )}} . \qquad Equation 1$$

Where v is the propagation velocity, E is the elasticity modulus, p is the density of the medium and u is the Poisson's ratio.
In addition, sound can be regarded as a time domain signal, and expressed as a sum of many sinusoidal signals with different frequencies, amplitudes, and phases. Therefore, the sound signal is often converted from the time domain into the frequency domain by the Fourier transform using:

$$F ( f ) = {\sum }_{- ∞}^{∞} f ( t ) {e}^{- j 2 π f t} dt . \qquad Equation 2$$

Where f (t) is the time domain sound signal, f is the frequency, and F(f) is the Fourier transform of f (t). Then, the amplitude spectrum, and the power spectrum of F(f), |F(f)| and P(f) can be acquired by Eq. 3 and 4, respectively. The specific features of |F(f)| and P(f) are related to the internal structure and physical properties of the transmission media, such as the highest peak and the centroid location of |F(f)|, the area of P(f) etc.

$$\vert F ( f ) \vert = {[ [ ( {\int }_{- w}^{4 w} f ( t ) {cos ( 2 π f ( t ) dt )}^{2} ] + [ {\int }_{- w}^{4 w} f ( t ) {sin ( 2 π f ( t ) dt )}^{2} ] ]}^{1 / 2} . \qquad Equation 3$$

$$P ( f ) = {\vert F ( f ) \vert}^{2} = {[ {[ ( {\int }_{- w}^{4 w} f ( t ) cos ( 2 π f ( t ) dt ) ]}^{2} + {[ {\int }_{- w}^{4 w} f ( t ) sin ( 2 π f ( t ) dt ) ]}^{2} ]}^{1} . \qquad Equation 4$$

To indicate firmness for spherical fruits, the stiffness coefficient (SC) can be calculated as:

$$S C = {m}^{2 / 3} {f}^{2} . \qquad Equation 5$$

Where SC is stiffness coefficient, f is the dominant resonant frequency where the response magnitude is greatest (Hz) and m the fruit mass (g).

$$E C = {m}^{2 / 3} {f}^{2} {ρ}^{1 / 3} . \qquad Equation 6$$

Where EC is the elasticity coefficient, ρ the density.

Acoustic vibration technology is a field of study that uses acoustic waves to measure and analyze the properties of materials and structures. Acoustic waves are generated by vibrating objects, and they travel through materials and structures. When they hit an object, they are reflected, refracted, or absorbed. The way that acoustic waves interact with an object can be used to determine its properties, such as its density, stiffness, and strength.

Acoustic vibration technology is used in a wide variety of applications, including:
Nondestructive testing: Acoustic vibration technology can be used to detect flaws in materials and structures without damaging them.
Quality control: Acoustic vibration technology can be used to monitor the quality of products during manufacturing.
Structural health monitoring: Acoustic vibration technology can be used to monitor the condition of bridges, buildings, and other structures.
Sound and vibration control: Acoustic vibration technology can be used to reduce noise and vibration in machines and vehicles.
The acoustic vibration of a plant can be used to determine the water stress of the plant. Water stress is a condition that occurs when a plant does not have enough water. The plant will wilt, and its leaves will droop. The acoustic vibration of the plant will also decrease when it is under water stress. There are a few different ways to measure acoustic vibration. One way is to use a microphone to record the sound of the plant vibrating. Another way is to use a sensor to measure the vibrations of the plant. Acoustic vibration can also be used to measure the moisture content of soil, and to identify the presence of water stress in hydroponic plants. The acoustic velocity of the tomatoes was correlated with their moisture content. The tomatoes with a lower acoustic velocity had a lower moisture content and were more likely to be under water stress.

Various techniques use acoustic vibrations for the nondestructive evaluation of agricultural products. Some of the various techniques used acoustic vibrations for the nondestructive evaluation of agricultural products are:
Ultrasonic vibration. Ultrasonic vibration is a high-frequency vibration that can be used to measure the properties of agricultural products, such as their density, stiffness, and moisture content. These properties are related to the quality of the agricultural products.
   Ultrasound can be used to measure the shelf life of fruits and vegetables by measuring the following properties:
Firmness: Ultrasound can measure the firmness of a fruit or vegetable by measuring the amount of force required to penetrate the surface. The firmer the fruit or vegetable, the more force will be required to penetrate the surface.
Ripeness: Ultrasound can measure the ripeness of a fruit or vegetable by measuring the amount of water that is present in the tissue. The riper the fruit or vegetable, the more water will be present in the tissue.
Texture: Ultrasound can measure the texture of a fruit or vegetable by measuring the amount of air that is present in the tissue. The more air that is present in the tissue, the more porous and soft the fruit or vegetable will be.
   Ultrasound can also be used to measure the shelf life of fruits and vegetables by measuring the rate at which the fruits and vegetables lose water. The faster the fruits and vegetables lose water, the shorter their shelf life will be.
Acoustic resonance. Acoustic resonance is the vibration of an object at a specific frequency. The frequency of resonance is determined by the size and shape of the object. Acoustic resonance can be used to identify the type of agricultural product.
Acoustic emission. Acoustic emission (AE) is the generation of sound waves when a material is stressed. The amount of acoustic emission is related to the amount of stress on the material. Acoustic emission can be used to detect defects in agricultural products. AE can be used to detect a variety of plant diseases, including:
   Bacterial wilt
   Botrytis blight
   Canker
   Crown gall
   Fusarium wilt
   Leaf spot
   Powdery mildew
   Rust
   Scab
   Stem rot
   Verticillium wilt
      AE is a non-destructive testing method, which means that it does not damage the plant. This makes it a valuable tool for early detection of plant diseases, when treatment is most effective.
      AE can be used to detect diseases in plants at a very early stage, often before any visible symptoms are present. This allows growers to take action to prevent the spread of the disease and to protect their crops.
Acoustic microscopy. Acoustic microscopy is a technique that uses sound waves to image the surface of objects. Acoustic microscopy can be used to inspect the surface of agricultural products for defects.
Acoustic spectroscopy. Acoustic spectroscopy is a technique that uses sound waves to identify the chemical composition of objects. Acoustic spectroscopy can be used to identify the type of agricultural product.
Acoustic vibrations can also be used to develop a 3D image of a fruit by using a technique called acoustic tomography. Acoustic tomography is a non-destructive imaging technique that uses sound waves to create an image of the interior of an object.
To create an acoustic image of a fruit, a sound wave is emitted from a transducer and propagates through the fruit. The sound wave is then reflected back to the transducer from the surfaces of the fruit. The time it takes for the sound wave to travel through the fruit and return to the transducer is used to calculate the distance to the surface of the fruit.
The distance to the surface of the fruit is then used to create an image of the fruit, with each pixel in the image representing the distance to a different surface of the fruit. The image can then be used to identify the different parts of the fruit, such as the core, the skin, and the flesh.
Acoustic tomography is a powerful technique that can be used to create detailed images of the interior of fruits. This information can be used to improve the quality of fruits, such as by identifying ripe fruits or by detecting defects in fruits.
   These techniques are non-destructive, which means that they do not damage the agricultural products. They are also rapid and accurate, which makes them ideal for the inspection of large quantities of agricultural products.

The methods are also classified according to sensors for vibration detection and excitation methods. There are two kinds of sensors: contact and noncontact sensors. Contact sensors are directly attached to the surface of the sample under examination. Such sensors that are commonly used include acceleration pickups and piezoelectric sensors. Noncontact sensors include microphones and optical sensors such as laser Doppler vibrometers (LDVs) and laser interferometers. The advantages of noncontact sensors are that they are totally nondestructive and exert no physical or mechanical influence; therefore, they do not damage the surface of a sample. In the case of contact sensors, the mass of the sensors must be negligibly small compared with the mass of the specimen. Another factor is mechanical excitation methods. Such methods are categorized as impact methods or forced vibration methods. Impact methods provide an instantaneous force on a sample, which freely vibrates after excitation. Any striking tool such as a metal or wooden stick may be used for excitation. A problem is that it is difficult to provide a constant excitation force when repeating the measurement; however, a pendulum is able to give an almost constant force if it is released at the same height. These methods cannot monitor the excitation force (input signal), which is needed to obtain a transfer function (especially the phase difference between input and output signals), unless using a tool such as an impact hammer equipped with an excitation force detector. While the use of a pendulum is effective, it is not practical in a field environment. Forced vibration methods continuously provide a varying (constant on average) force on a sample during measurement. These methods usually use an electrodynamic shaker or a piezoelectric vibrator. Forced vibration methods are advantageous in terms of their repeatability when compared with impact methods. In addition, the methods provide a transfer function by monitoring excitation force. The methods are classified further according to the excitation force as methods employing random or swept sine wave excitations. The random excitation method usually uses a force with a wide frequency band component. The swept sinc method provides a force with a gradually increasing or decreasing frequency within a certain frequency range. The swept sine wave method also called as a chirp, provides more effective excitation for accurate determination of the resonance of the samples than the random excitation method because it provides excitation energy concentrated in a small frequency band and is able to provide reproducible and repeatable measurements, and the excitation energy is spread over frequencies of a wide range in a limited time period. Usually for a sinc sweep method, a vibration shaker pan is needed to be affective, however in this case we are using the bone technology based speakers that vibrate the fruit/object in contact, allowing the piezoelectric sensors to capture the natural resonance of the objects.

Acoustic vibrations can be used to kill germs in food and perishables. This is a process called sonication, and it works by creating high-frequency vibrations that disrupt the cell walls of bacteria and other microorganisms. This process can be used to sanitize food surfaces, as well as to kill bacteria in food products like milk and meat.

Sonoporation is a technique that uses sound waves to increase the permeability of cell membranes. This can be used to kill bacteria by allowing antibiotics or other drugs to enter the cells and kill them.
Ultrasonication is a process that uses high-frequency sound waves to create cavitation bubbles. These bubbles can collapse and release energy, which can damage bacteria and other cells.
Sonochemistry is the study of the effects of sound waves on chemical reactions. This can be used to kill bacteria by using sound waves to increase the rate of chemical reactions that kill bacteria.
Sonoluminescence is the emission of light from a cavitation bubble. This light can be used to kill bacteria by damaging their DNA.
Sonoporation, ultrasonication, sonochemistry, and sonoluminescence are all methods that can be used to kill bacteria using acoustic vibrations. These methods are effective and safe, and they can be used to sanitize food surfaces and to kill bacteria in food products.

Acoustic vibrations can also be used to do a quality check on pharmaceutical drugs. This is a non-destructive testing method that can be used to identify defects in tablets, capsules, and other solid dosage forms. to conduct a quality check on pharmaceutical drugs. The basic idea is to use sound waves to probe the drug particles and measure their physical properties. This information can then be used to identify any impurities or defects in the drug. Acoustic vibrations can also be used to detect cracks. This is a non-destructive testing (NDT) method that can be used to identify cracks in a variety of materials, including metals, plastics, and composites. The acoustic vibration testing method works by applying a controlled vibration to the material. The vibration causes the material to vibrate at its natural frequency. Any cracks in the material will cause it to vibrate at a different frequency. This difference in frequency can be detected by a sensor, which can then be used to identify the crack.

Acoustic vibrations can be used for drug delivery in a variety of ways. One way is to use ultrasound to break up drug-containing particles into smaller particles that can be more easily absorbed by the body. Another way is to use ultrasound to create microbubbles in the bloodstream. These microbubbles can then be used to deliver drugs directly to tumors or other diseased tissues.

### SUMMARY

In accordance with the purpose(s) of the invention, as embodied and broadly described herein, this disclosure, in one aspect, relates to acoustic method of predicting the shelf life of perishables using a combination of audio analysis, temperature and humidity data, bruising intensity, and overall browning index. The acoustic vibration of fruits changes with maturity due to the changes in the cell structure of the fruit. As the fruit ripens, the cells become softer and the fruit becomes more fragile. This causes the fruit to vibrate more easily and produce a higher-pitched sound.

Acoustic vibration technology has been used to assess the maturity of fruits such as apples, tomatoes, and bananas. The technology involves placing a sensor on the fruit and measuring the vibrations produced by the fruit. The vibrations are then analyzed to determine the maturity of the fruit.
Acoustic vibration technology is a non-destructive method of assessing fruit maturity. This means that the fruit does not have to be damaged in any way to be assessed. Acoustic vibration technology is also a rapid method of assessing fruit maturity.
Normal sound waves are tiny vibrations in the air. The vibrations travel through the air to our cardrums. The cardrums in turn vibrate, decoding these sound waves into a different type of vibrations that are received by the Cochlea, also known as the inner ear. The Cochlea is connected to our auditory nerve, which transmits the sounds to our brain.

Bone conduction technology is another method of transmitting sound waves through the bones of the skull to the inner ear. This is in contrast to traditional headphones, which transmit sound waves through the ear canal.

In the present invention, a bone technology-based speaker system is used to determine acoustic vibrations of perishable commodities by placing the speaker on the fruit and monitoring the sound waves that are transmitted through the fruits. The sound waves will vary depending on the ripeness of the fruit. A ripe fruit will have a higher natural resonance frequency than an unripe fruit. This is because the ripe fruit is softer and more flexible, which allows it to vibrate at a higher frequency. The natural resonance frequency of a fruit can be used to determine its ripeness. A ripe fruit will have a higher natural resonance frequency than an unripe fruit. This is because the ripe fruit is softer and more flexible, which allows it to vibrate at a higher frequency.
Bone conduction speakers can also be used to determine the acoustic vibrations of other objects, such as vegetables, beverages, seeds and flowers. This information can be used to improve the quality of fruits and vegetables, as well as to develop new products.

A piezoelectric microphone is a type of microphone that uses the piezoelectric effect to convert sound waves into an electrical signal. The piezoelectric effect is the ability of certain materials to generate an electric charge when they are subjected to mechanical stress. In a piezoelectric microphone, the diaphragm is made of a piezoelectric material, and when sound waves hit the diaphragm, they cause it to vibrate. This vibration causes the piezoelectric material to generate an electric signal, which is then amplified and converted into an audio signal.

Piezoelectric microphones are often used in applications where there is a lot of noise, such as in industrial settings or in outdoor environments. They are also used in some types of musical instruments, such as the electric guitar.
Piezoelectric microphones have several advantages over other types of microphones. They are very sensitive to sound, and they can be made very small. They are also very durable, and they can withstand a lot of vibration. Piezoelectric films are useful devices for sensing vibrations within agricultural products. In the present invention, a piezoelectric microphone is placed in proximity to the fruit and is used to ‘listen’ to the vibrations (generated by the swept sine wave method) traveling through the fruit.

In the present invention a bone technology based sensor with a piezoelectric microphone can be used to measure the acoustic vibrations of fruits by placing the sensor on the fruit and recording the vibrations. The vibrations can then be analyzed to determine the ripeness of the fruit.

Bone technology based sensors are made from materials that are similar to the bones in the human body. These materials are piezoelectric, which means that they generate an electrical signal when they are subjected to mechanical stress. This electrical signal can be used to measure the vibrations of the fruit.
Piezoelectric microphones are also made from piezoelectric materials. These microphones can convert sound waves into electrical signals. The electrical signals from the microphone can be used to record the vibrations of the fruit.
The vibrations of the fruit can be analyzed to determine the ripeness of the fruit. Ripe fruits will vibrate at a higher frequency than unripe fruits. The frequency of the vibrations can also be used to determine the mass of the fruit. Ripe fruits will have a higher mass than unripe fruits.
The information from the vibrations of the fruit can be used to improve the quality of fruits. For example, the information can be used to determine the best time to pick fruits. The information can also be used to determine the best way to store fruits.

### BRIEF DESCRIPTION OF THE DRAWINGS

Reference is made to the accompanying drawings in which is shown an illustrative embodiment of the invention, from which its novel features and advantages will be apparent.

FIG. 1 is a simplified illustration of the Acoustic Method to predict the maturity and the Remaining Shelf-Life Prediction methodology.

FIG. 2 is a simple illustration of the bone technology-based speakers.

FIG. 3 is an illustration of the piezoelectric based microphones.

### DESCRIPTION OF THE PREFERRED EMBODIMENTS

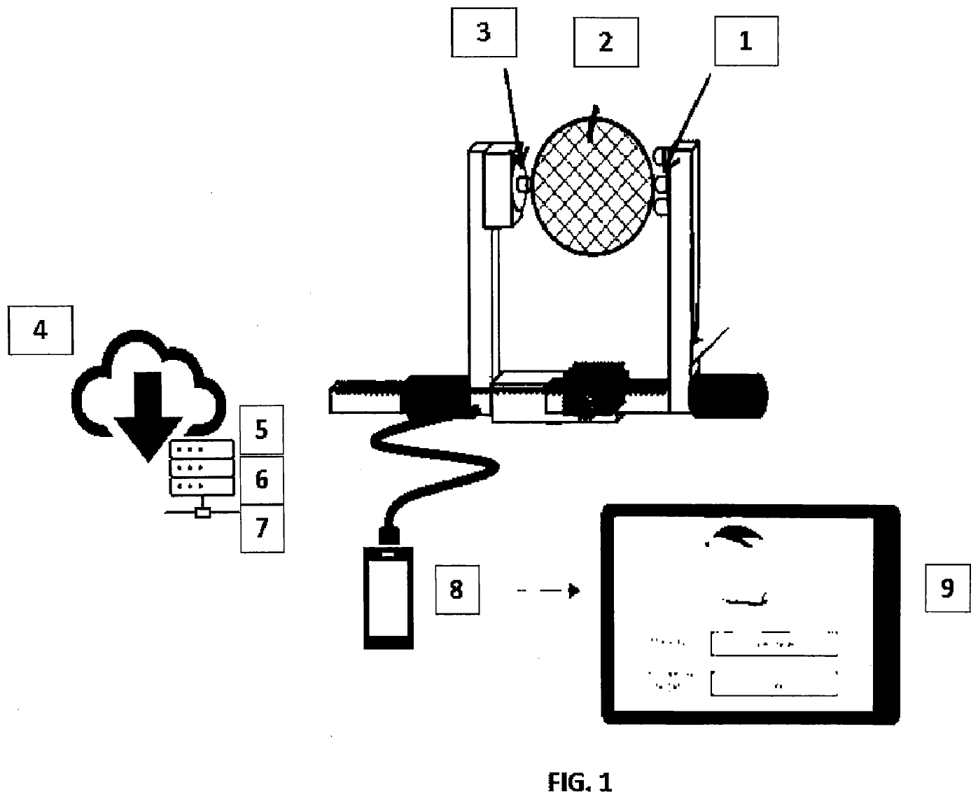

Referring to FIG. 1, it will be seen that an illustrative includes there is produce 2 at a distribution/retail center, which is placed between a clamp like structure (eventually it would be a glove), which is embedded with a bone conduction speaker 1 on one side, and a piezoelectric microphone 3 on the other side. A swept sine wave method is loaded on 1, and the response is measured on 3. A vibration data of the produce is captured, via the sensor, and is compared with the audio files resides on the cloud server 4. The cloud server has three audio matrices, 5 Shelf-Life Matrix, 6 Defect Matrix, and 7 is the Maturity Matrix either resides on the cloud server or on the audio device itself, 8 is the analysis device (either a phone, tablet or a computer). 9 is the results panel which documents the maturity stage and remaining shelf life of the produce.

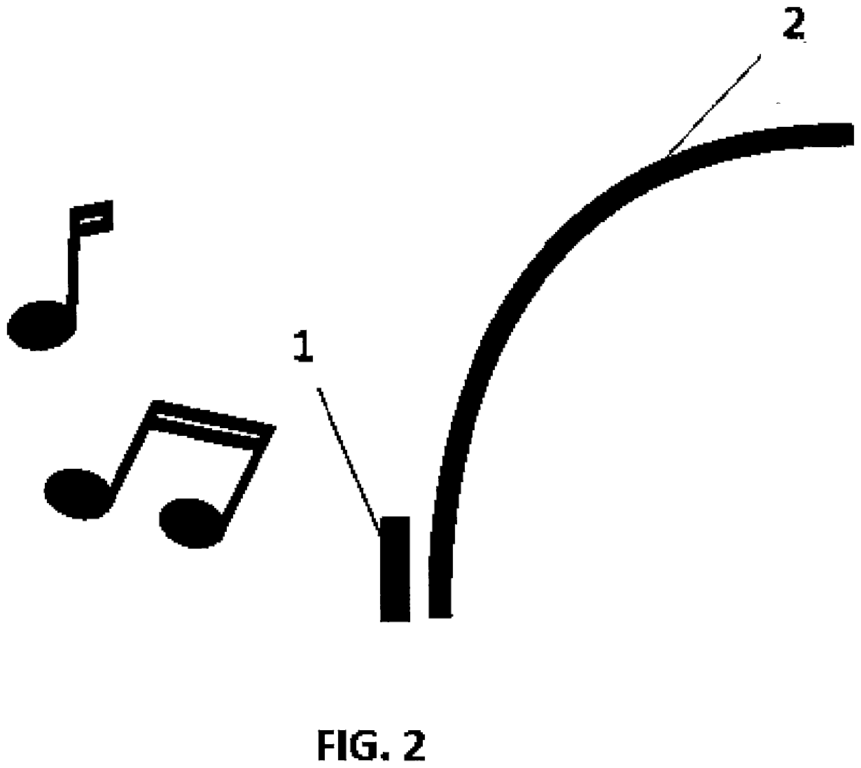

Referring to FIG. 2, it will be seen that an illustrative includes a bone conduction speaker 1 placed in contact with the fruit/produce 2.

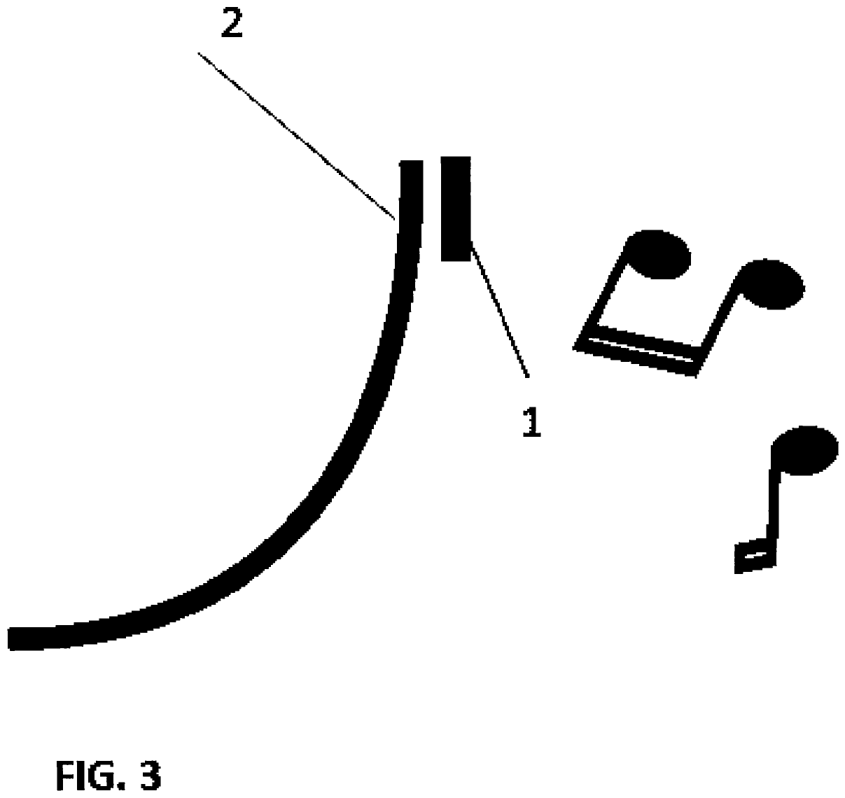

Referring to FIG. 3, it will be seen that an illustrative includes a piezoelectric sensor-based microphone 1 placed in contact with the fruit/produce 2.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

As used in the specification and the appended claims, the singular forms “a,” “an” and “the” include plural referents unless the context clearly dictates otherwise.

In one aspect, the present disclosure provides a method for predicting the ready for harvest condition of fruits and vegetables, the method comprising generating an image of the said fruits/vegetables, resizing and cropping the image, separating the red, green and blue

What is claimed is:

1. A Shelf life Prediction System including a bone conductor based speaker having a sound frequency range of at least 1 hz and at most 20K hz; A piezo electric sensor based microphone having a sound frequency range of at least 1 hz and at most 20K hz, for predicting the remaining shelf life of fruits and vegetables, the steps comprising:
Generating an acoustic vibration profile of said fruit/vegetable; Resizing and cropping the audio to the area of interest; comparing the data with the combination of acoustic vibrations based shelf life, defect and maturity matrices; finding the match between the acoustic vibration space and the acoustic vibration space from the shelf life, defect and matrices, predicting the remaining shelf life based upon the matching process.

2. A Shelf life Prediction System including a bone conductor based speaker having a sound frequency range of at least 1 hz and at most 20K hz; A bone conductor sensor based microphone having a sound frequency range of at least 1 hz and at most 20K hz, for predicting the ready for harvest condition of fruits and vegetables, the steps comprising:
Generating an acoustic vibration profile of said fruit/vegetable; Resizing and cropping the audio to the area of interest; comparing the data with the combination of acoustic vibrations-based shelf life, defect and maturity matrices; finding the match between the acoustic vibration space and the acoustic vibration space from the shelf life, defect and maturity matrices, predicting the remaining shelf life based upon the matching process.

3. A prediction method of claim 1, further configured to integrate into permanently affixed refrigerated/non-refrigerated drawers and/or cabinets.

4. A method of claim 2, further configured to integrate into robotic arm, equipped with handgrip mechanism to allow for automated harvesting and pruning.

5. A Detection System including a piezo electric based speaker having a sound frequency range of at least 1 hz and at most 20K hz; A piezo electric sensor based microphone having a sound frequency range of at least 1 hz and at most 20K hz, for using acoustic vibrations for detecting diseases in plants:
Generating an acoustic vibration profile of said plant; Resizing and cropping the audio to the area of interest; comparing the data with the combination of acoustic vibrations based based diseases, defect and maturity matrices; finding the match between the acoustic vibration space and the acoustic vibration space from the diseases, defect and maturity matrices, detecting the disease based upon the matching process.

6. A method of claim 5, further configured to integrate into robotic arm, equipped with handgrip mechanism to allow for automated disease detection.
